(19) United States
(12) Patent Application Publication
(10) Pub. No.: US 2023/0288320 A1
(43) Pub. Date: Sep. 14, 2023

(54) DETECTION APPARATUS AND MEASURING APPARATUS

(71) Applicant: SEIKO EPSON CORPORATION, Tokyo (JP)

(72) Inventor: Takashi TAJIRI, MATSUMOTO-SHI (JP)

(73) Assignee: SEIKO EPSON CORPORATION, Tokyo (JP)

(21) Appl. No.: 18/179,380

(22) Filed: Mar. 7, 2023

(30) Foreign Application Priority Data
Mar. 8, 2022 (JP) 2022-035057

(51) Int. Cl. G01N 21/21

(52) U.S. Cl. CPC G01N 21/21

(57) ABSTRACT

A measuring apparatus identifies biological information from a detection signal of a detection apparatus. The detection apparatus includes a light emitting unit and a light receiving unit aligned in an X direction, and a polarizer that covers a +Z direction, which is an incident side of light in the light receiving unit. The polarizer is a light absorbing polarizer having an absorption axis with an axial direction matching a Y direction, absorbs an s-polarized component of light in the light receiving unit, and transmits a p-polarized component.

The present application is based on, and claims priority from JP Application Serial Number 2022-035057, filed Mar. 8, 2022, the disclosure of which is hereby incorporated by reference herein in its entirety.

### BACKGROUND

### 1. Technical Field

The present disclosure relates to a detection apparatus and a measuring apparatus including a light emitting unit and a light receiving unit.

### 2. Related Art

Various measurement technologies for non-invasively measuring biological information such as heartbeats have been proposed in the related art. JP 2001-353133 A describes a detection apparatus (detector) including a light emitting unit configured to emit light toward a living body, and a light receiving unit configured to receive light reflected by the living body. Biological information can be acquired by analyzing a signal output from the light receiving unit of this type of detection apparatus. For example, JP 2001-353133 A describes a pulse wave sensor configured to measure a pulse rate from a signal output from the light receiving unit.

In JP 2001-353133 A, a front surface of a light transmissive plate covering the light emitting unit and the light receiving unit is formed as a protruding curved surface, and thus adhesion between the light transmissive plate and skin is improved. This makes it possible to prevent light emitted from the light emitting unit from being reflected by a skin surface, and becoming stray light incident directly on the light receiving unit. Furthermore, disturbance light can be prevented from entering the light receiving unit. Therefore, it is possible to prevent light not including biological information from entering the light receiving unit, thereby improving signal accuracy.

Although the configuration disclosed in JP 2001-353133 A can suppress stray light and disturbance light, the light reflected at the skin surface and entering the light receiving unit cannot be eliminated. Furthermore, it is not possible to prevent the light emitted from the light emitting unit from being reflected by a back surface of the light transmissive plate (a front surface on the light receiving unit side) and directly entering the light receiving unit.

### SUMMARY

In order to solve the problem described above, a detection apparatus of the present disclosure includes a light emitting unit; a light receiving unit aligned with the light emitting unit in a first direction, and a polarizer configured to cover at least one of an emission side of light in the light emitting unit, and an incident side of light in the light receiving unit, wherein the polarizer absorbs or reflects an s-polarized component.

A measuring apparatus of the present disclosure includes the detection apparatus described above, and an information analysis unit configured to identify biological information from a detection signal indicating a detection result of the detection apparatus.

### BRIEF DESCRIPTION OF THE DRAWINGS

FIG. 1 is a side view of a measuring apparatus of a first exemplary embodiment.

FIG. 2 is a block diagram illustrating a functional configuration of the measuring apparatus of the first exemplary embodiment.

FIG. 3 is a plan view of a detection apparatus of the first exemplary embodiment.

FIG. 4 is a cross-sectional view schematically illustrating a cross-sectional configuration of the detection apparatus of the first exemplary embodiment.

FIG. 5 is a diagram schematically illustrating a situation when detecting light including biological information using the detection apparatus of the first exemplary embodiment.

FIG. 6 is a diagram illustrating a situation when light including s-polarized light and p-polarized light is incident on a boundary surface between materials having different refractive indices.

FIG. 7 is formulae and a graph illustrating a relationship between incident angle on the boundary surface between the materials having the different refractive indices, and reflectance of the s-polarized light and reflectance of the p-polarized light.

FIG. 8 is a diagram schematically illustrating a cross-sectional configuration of a detection apparatus of a second exemplary embodiment, and a situation when detecting light including biological information.

FIG. 9 is a diagram schematically illustrating a cross-sectional configuration of a detection apparatus of a third exemplary embodiment, and a situation when detecting light including biological information.

FIG. 10 is a diagram schematically illustrating a cross-sectional configuration of a detection apparatus of a fourth exemplary embodiment.

FIG. 11 is a diagram schematically illustrating a cross-sectional configuration of a detection apparatus of a fifth exemplary embodiment.

### DESCRIPTION OF EXEMPLARY EMBODIMENTS

Exemplary embodiments of the present disclosure are described below with reference to the accompanying drawings. Note that in each of the drawings, each member is schematically illustrated in a size to be recognizable, and actual dimensions and ratios may be different from those illustrated in the drawings.

### 1. First Exemplary Embodiment Measuring Apparatus

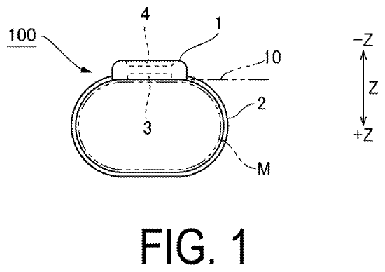

FIG. 1 is a side view of a measuring apparatus 100 of a first exemplary embodiment. The measuring apparatus 100 is a biometric measuring apparatus for non-invasively measuring biological information. The measuring apparatus 100 is used facing a site to be measured (hereinafter referred to as a “measuring site M”) in a subject's body (living body). In the example illustrated in FIG. 1, the measuring apparatus 100 is a wristwatch type portable device including a housing 1 and a belt 2. The measuring site M is a wrist of the subject. The measuring apparatus 100 is worn and used by winding the band-shaped belt 2 around the wrist of the subject, so that a detection surface 10 of the housing 1 faces a skin surface of the wrist (measuring site M).

In the present specification, an X direction, a Y direction, and a Z direction are directions orthogonal to each other. The X direction is a first direction. The Y direction is a second direction. The Z direction is a normal direction of the detection surface 10. A+Z direction is a direction from the detection surface 10 toward the measuring site M, and a −Z direction is a direction from the measuring site M toward the detection surface 10.

In the present specification, a heartbeat (for example, a pulse rate) and an oxygen saturation (SpO₂) of the subject are exemplified as biological information. The heartbeat indicates the change of the internal volume of a blood vessel over time due to the pulsation of the heart. The oxygen saturation indicates the proportion (%) of hemoglobin bound to oxygen in hemoglobin in the blood of the subject and is an index for evaluating the respiratory function of the subject.

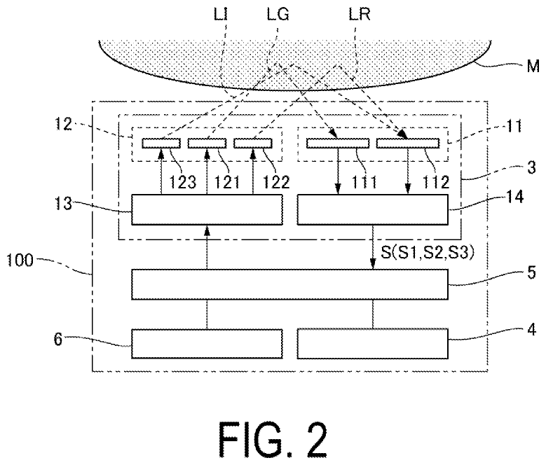

FIG. 2 is a block diagram illustrating a functional configuration of the measuring apparatus 100 of the first exemplary embodiment. As illustrated in FIG. 2, the measuring apparatus 100 includes a control device 5, a storage device 6, a display device 4, and a detection apparatus 3. The control device 5 and the storage device 6 are disposed inside the housing 1. The detection apparatus 3 is disposed at the detection surface 10. The display device 4 is disposed at a surface of the housing 1 on an opposite side to the detection surface 10. The display device 4 displays various images including measurement results under control of the control device 5. The display device 4 is, for example, a liquid crystal display panel.

Note that, in addition to the functional configuration illustrated in FIG. 2, the measuring apparatus 100 may be configured to include an operation unit such as an operation button or a touch panel disposed at the surface of the housing 1, and to input an operation signal in accordance with an operation for the operation unit to the control device 5. Furthermore, a communication unit configured to output a measurement result to an outside and input a signal from the outside to the control device 5 may be included. Alternatively, a voice output unit or a vibration unit may be included as a device for notifying of a measurement result.

As illustrated in FIG. 2, the detection apparatus 3 includes a light receiving unit 11, a light emitting unit 12, a drive circuit 13, and an output circuit 14. One or both of the drive circuit 13 and the output circuit 14 can also be installed as circuits external to the detection apparatus 3. That is, the drive circuit 13 and the output circuit 14 may be omitted from the detection apparatus 3.

As illustrated in FIG. 1, the detection apparatus 3 is disposed at the detection surface 10. The detection apparatus 3 is a reflective optical sensor module that emits light from the detection surface 10, receives light incident on the detection surface 10 from the measuring site M, and generates detection signals S. As illustrated in FIG. 2, the light emitting unit 12 includes a first light emitting element 121, a second light emitting element 122, and a third light emitting element 123. The first light emitting element 121, the second light emitting element 122, and the third light emitting element 123 emit light having different wavelengths from the detection surface 10, respectively. For the first light emitting element 121, the second light emitting element 122, and the third light emitting element 123, LEDs are used, for example.

The first light emitting element 121 emits first light LG. The first light LG is, for example, green light having a green wavelength band from 520 nm to 550 nm, and is light having a peak wavelength of 520 nm. The second light emitting element 122 emits second light LR. The second light LR is, for example, red light having a red wavelength band from 600 nm to 800 nm, and is light having a peak wavelength of 660 nm. The third light emitting element 123 emits third light LI. The third light is, for example, near-infrared light having a near-infrared wavelength band from 800 nm to 1300 nm. The third light LI is, for example, light having a peak wavelength of 905 nm. Note that, the wavelengths of light emitted by the respective light emitting elements are not limited to the above wavelength bands.

The drive circuit 13 supplies a drive current to cause each of the first light emitting element 121, the second light emitting element 122, and the third light emitting element 123 to emit light. For example, the drive circuit 13 periodically causes each of the first light emitting element 121, the second light emitting element 122, and the third light emitting element 123 to emit light in a time-division manner. The light emitted from each of the first light emitting element 121, the second light emitting element 122, and the third light emitting element 123 is incident on the measuring site M from the detection surface 10, propagates while repeatedly reflecting and scattering inside the measuring site M, then is emitted from the measuring site M, and is incident on the light receiving unit 11 disposed at the detection surface 10.

As illustrated in FIG. 2, the light receiving unit 11 includes a first light receiving unit 111 and a second light receiving unit 112. Each of the first light receiving unit 111 and the second light receiving unit 112 generates a detection signal according to intensity of received light. Photodiodes or photo transistors are used for the first light receiving unit 111 and the second light receiving unit 112.

The first light receiving unit 111 receives the first light LG that has propagated in the measuring site M after being emitted from the first light emitting element 121 and generates a detection signal according to intensity of the received light. The second light receiving unit 112 receives the second light LR that has propagated in the measuring site M after being emitted from the second light emitting element 122 or the third light LI that has propagated in the measuring site M after being emitted from the third light emitting element 123 and generates detection signals according to intensity of the received light.

The first light receiving unit 111 includes a band-pass filter that selectively transmits the light of the wavelength emitted from the first light emitting element 121 (that is, the green light). As described below, the first light receiving unit 111 is disposed at a position close to the light emitting unit 12 from the second light receiving unit 112, and thus, receives a large amount of the first light LG (green light) that propagates by a short distance in the living body and then returns. The first light receiving unit 111 blocks the second light LR (red light) and the third light LI (near-infrared light) by the band-pass filter, and receives the first light LG (green light).

A distance by which the first light LG (green light) can propagate through the living body is short, and thus is sufficiently attenuated before reaching the second light receiving unit 112. Therefore, in the second light receiving unit 112, a large amount of the second light LR (red light) or the third light LI (near-infrared light) is received. Note that, the second light receiving unit 112 may include a band-pass filter that selectively receives the second light LR (red light) and the third light LI (near-infrared light).

The first light receiving unit 111 and the second light receiving unit 112 may each include an angle limiting filter. The angle limiting filter transmits light incident at an angle smaller than an allowable incident angle and blocks light incident at an angle larger than the allowable incident angle. Accordingly, in each of the first light receiving unit 111 and the second light receiving unit 112, outside light incident from a direction different from the measuring site M of the living body can be blocked.

The output circuit 14 is configured to include, for example, an A/D converter that converts detection signals generated by each of the first light receiving unit 111 and the second light receiving unit 112 from analog to digital and an amplifier circuit that amplifies the converted detection signals (both not illustrated) and generates the plurality of detection signals S (S1, S2, S3) corresponding to wavelengths different from each other.

The detection signal S1 is a signal indicating the intensity of light received by the first light receiving unit 111 when it has received the first light LG (green light) emitted from the first light emitting element 121. The detection signal S2 is a signal indicating the intensity of light received by the second light receiving unit 112 when it has received the second light LR (infrared light) emitted from the second light emitting element 122. The detection signal S3 is a signal indicating the intensity of light received by the second light receiving unit 112 when it has received the third light LI (near-infrared light) emitted from the third light emitting element 123.

Each detection signal S is a heartbeat signal including periodic fluctuations corresponding to pulsations (volume heartbeats) of the artery inside the measuring site M because the amounts of absorption by blood during dilation and contraction of blood vessels generally differ.

The drive circuit 13 and the output circuit 14 are mounted at a substrate in a form of IC chips, for example, together with the light receiving unit 11 and the light emitting unit 12. Note that, the drive circuit 13 and the output circuit 14 can be installed as external circuits of the detection apparatus 3 as described above.

The control device 5 is an arithmetic processing unit such as a central processing unit (CPU) or a field-programmable gate array (FPGA) and controls the entirety of the measuring apparatus 100. The storage device 6 includes, for example, a non-volatile semiconductor memory and stores a program executed by the control device 5 and various data used by the control device 5. Note that, a configuration may be adopted in which the functions of the control device 5 are distributed over a plurality of integrated circuits or a configuration in which some or all of the functions of the control device 5 are realized by a dedicated electronic circuit. Note that, although the control device 5 and the storage device 6 are illustrated as separate elements in FIG. 2, the control device 5 including the storage device 6 can also be realized, for example, by an ASIC, or the like.

The control device 5 identifies biological information of the subject from the plurality of detection signals S (S1, S2, S3) generated by the detection apparatus 3 by executing the program stored in the storage device 6. Specifically, the control device 5 can identify a heartbeat of the subject from the detection signal S1 indicating the intensity of first light LG (green light) received by the first light receiving unit 111. The control device 5 can identify a pulse rate of the subject based on, for example, the detection signal S1. Further, the control device 5 can also identify the oxygen saturation (SpO₂) of the subject by analyzing the detection signal S2 indicating the intensity of second light LR (red light) received by the light receiving unit 11, and the detection signal S3 indicating the intensity of third light LI (near-infrared light) received by the light receiving unit 11.

The control device 5 functions as an information analysis unit that identifies the biological information from the detection signals S indicating the detection results of the detection apparatus 3 as described above. The control device 5 (information analysis unit) causes the display device 4 to display the biological information identified from the detection signals S. It is also possible to notify the user of the measurement result by voice output. It is also possible to notify the user of a warning (possibility of impaired physical function) when the pulse rate or oxygen saturation has fluctuated to values out of a predetermined range.

Detection Apparatus

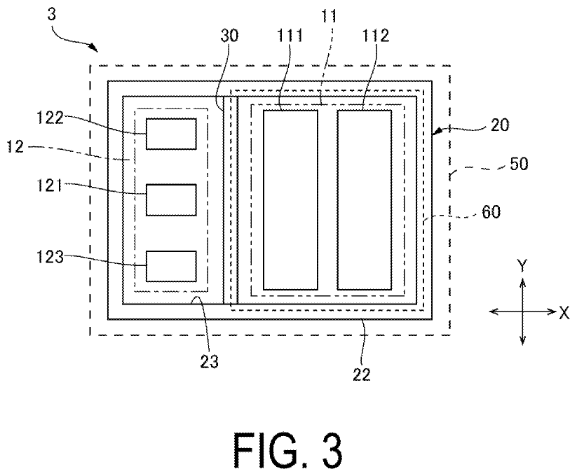

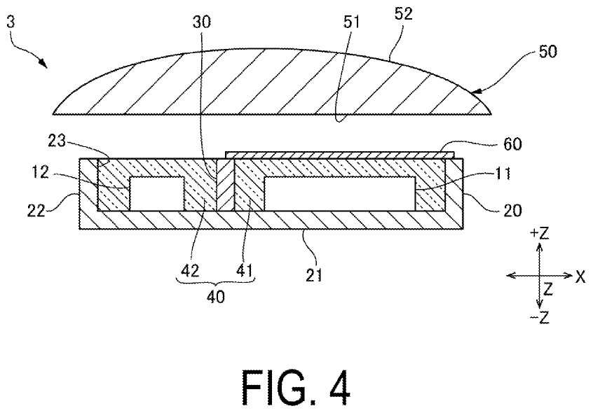

FIG. 3 is a plan view of the detection apparatus 3 of the first exemplary embodiment. FIG. 4 is a cross-sectional view schematically illustrating a cross-sectional configuration of the detection apparatus of the first exemplary embodiment, and illustrates a cross-sectional configuration cut along an XZ plane. As illustrated in FIGS. 3 and 4, the detection apparatus 3 includes the light receiving unit 11 and the light emitting unit 12, a case 20 that accommodates the light receiving unit 11 and the light emitting unit 12 in a state of being mounted at a substrate (not illustrated), a shield plate 30 disposed between the light receiving unit 11 and the light emitting unit 12, a sealing material 40 that seals (molds) the light receiving unit 11 and the light emitting unit 12, a cover 50 that covers the light receiving unit 11 and the light emitting unit 12, and a polarizer 60 that is disposed between the light receiving unit 11 and the cover 50.

As illustrated in FIGS. 3 and 4, the case 20 includes a rectangular base plate portion 21 with the X direction as a long side direction, and a side plate portion 22 protruding in the +Z direction from an outer edge of the base plate portion 21. The light receiving unit 11 and the light emitting unit 12 are fixed to the base plate portion 21 via a substrate (not illustrated), for example. The light emitting unit 12 emits light in the +Z direction, and the light receiving unit 11 receives light incident from the +Z direction.

The side plate portion 22 surrounds an outer periphery side of the light receiving unit 11 and the light emitting unit 12. The shield plate 30 shields light emitted from the light emitting unit 12 from directly entering the light receiving unit 11. In the first exemplary embodiment, the light receiving unit 11 and the light emitting unit 12 are aligned in the X direction. The shield plate 30 is disposed between the light receiving unit 11 and the light emitting unit 12, and extends in the Y direction. An inner peripheral surface of the side plate portion 22 and a front surface of the shield plate 30 are colored in black, for example, to suppress reflection.

As illustrated in FIG. 3, the light emitting unit 12 is a light emitting unit that includes the first light emitting element 121, the second light emitting element 122, and the third light emitting element 123 aligned in the Y direction. The first light emitting element 121 that emits the first light LG (green light) is disposed at a center in the Y direction of the light emitting unit 12. The second light emitting element 122 that emits the second light LR (red light) is disposed on one side in the Y direction of the first light emitting unit 121. The third light emitting element 123 that emits the third light LI (near-infrared light) is disposed on another side in the Y direction of the first light emitting unit 121.

The light receiving unit 11 is a light receiving unit that includes the first light receiving unit 111 and the second light receiving unit 112 aligned in the X direction. The first light receiving unit 111 that receives the first light LG (green light) is disposed at a position adjacent in the X direction to the light emitting unit 12. The second light receiving unit 112 that receives the second light LR (red light) and the third light LI (near-infrared light) is disposed on a side opposite to the light emitting unit 12 with respect to the first light receiving unit 111.

The sealing material 40 is an optically transparent resin material that is filled in an internal space of the case 20. In the present exemplary embodiment, the internal space of the case 20 is partitioned by the shield plate 30 into a space in which the light receiving unit 11 is disposed and a space in which the light emitting unit is disposed. Accordingly, the sealing material 40 includes a first sealing material 41 filled in the space in which the light receiving unit 11 is disposed, and a second sealing material 42 that is filled in the space in which the light emitting unit 12 is disposed.

The cover 50 is made of an optically transparent material such as glass or an optically transparent resin. The cover 50 covers an emission side of light in the light emitting unit 12, and an incident side of light in the light receiving unit 11. In the case 20 that accommodates the light emitting unit 12 and the light receiving unit 11, an end portion in the +Z direction of the side plate portion 22 is an opening portion 23, which is open in the +Z direction. The cover 50 is disposed in the +Z direction of the opening portion 23. An outer shape of the cover 50 viewed from the Z direction is larger than the opening portion 23, and the cover 50 covers the entire opening portion 23.

The cover 50 includes a first surface 51 facing the case 20 and the sealing material 40, and a second surface 52 facing an opposite side to the case 20 and the sealing material 40. The first surface 51 is a flat surface perpendicular to the Z direction. The second surface 52 is a protruding curved surface that is convex in the +Z direction. The first surface 51 does not contact front surfaces of the first sealing material 41 and the second sealing material 42, and faces the first sealing material 41 and the second sealing material 42 via a predetermined gap. The second surface 52 is disposed at a front surface of the housing 1 of the measuring apparatus 100, and functions as the detection surface 10.

The polarizer 60 is disposed between the cover 50 and the light receiving unit 11. The polarizer 60 is configured to absorb an s-polarized component of light incident from the cover 50 side, and to transmit a p-polarized component. For example, the polarizer 60 is a light absorbing polarizer having an absorption axis with an axial direction matching the Y direction.

The polarizer 60 is disposed at a front surface of the first sealing material 41. For example, the polarizer 60 is fixed to the front surface of the first sealing material 41 by bonding. The polarizer 60 has a shape when viewed in the Z direction that is substantially the same as or larger than the first sealing material 41, and covers an entire front surface in the +Z direction of the first sealing material 41. The polarizer 60 is, for example, a light absorbing polarizing plate made mainly with polyvinyl alcohol (PVA), by adsorbing and aligning iodine (I) compound molecules. Note that, it is sufficient that the polarizer 60 is configured to absorb the s-polarized component. For example, a wire grid polarizer using a light absorbing material can be used as the polarizer 60.

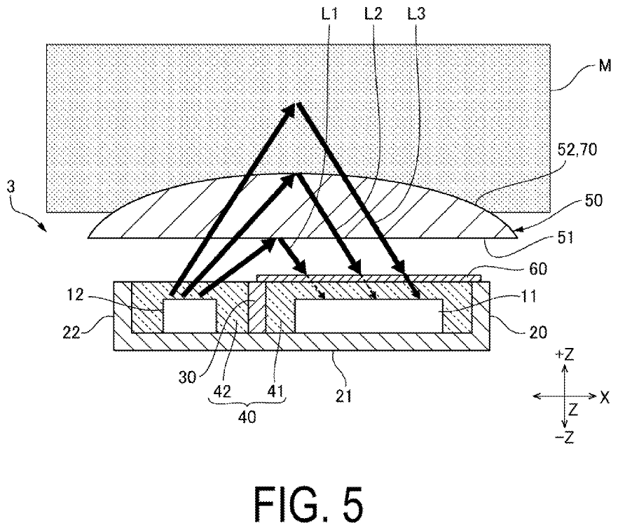

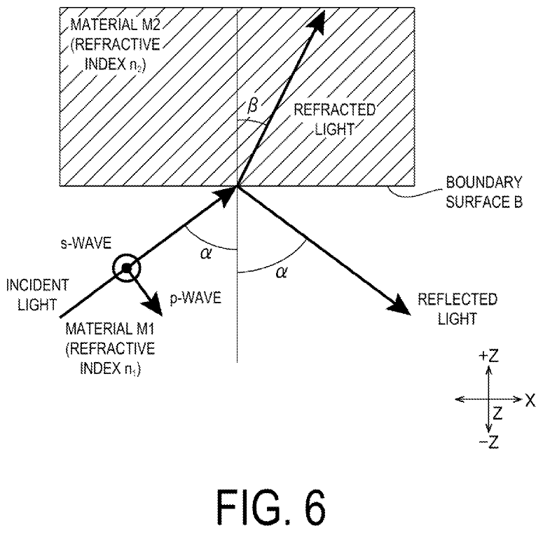

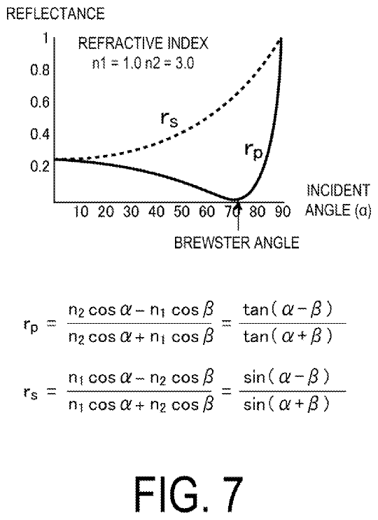

FIG. 5 is a diagram schematically illustrating a situation when detecting light including biological information using the detection apparatus 3 of the first exemplary embodiment. FIGS. 6 and 7 are diagrams for describing the principles of the present disclosure. FIG. 6 is a diagram illustrating a situation when light including s-polarized light (s-wave) and p-polarized light (s-wave) is incident on a boundary surface B between materials M1 and M2 having different refractive indices. FIG. 7 is formulae and a graph illustrating a relationship between incident angle α on the boundary surface B between the materials M1 and M2 having the different refractive indices, and reflectance rs of the s-polarized light and reflectance rp of the p-polarized light. The graph illustrated in FIG. 7 is a graph when a refractive index n1=1.0, and a refractive index n2=3.0.

As illustrated in FIG. 6, when light is incident on the boundary surface B between the material M1 (refractive index n1) and the material M2 (refractive index n2) having the different refractive indices, a part of the light is reflected. This reflection is referred to as Fresnel reflection. Further, the light is a wave, and is divided into the p-polarized light (p-wave) and the s-polarized light (s-wave), according to oscillating directions with respect to a reflective surface. At this time, the reflectance rp of the p-polarized light and the reflectance rs of the s-polarized light are determined by the incident angle α and an angle of refraction β with respect to the boundary surface B, as illustrated by the formulae in FIG. 7. As illustrated in FIG. 7, while intensity of reflected light of the p-polarized light (reflectance rp) decreases as the incident angle increases, once reaches 0, and increases, intensity of reflected light of the s-polarized light (reflectance rs) does not decrease as the incident angle, and increases continues to increase.

As can be seen from FIG. 7, when the light including the s-polarized light and the p-polarized light is reflected, the reflected light becomes light that has the large s-polarized component, and the small p-polarized component. For example, in an incident angle where the reflectance of the p-polarized light is 0 (this angle is referred to as a Brewster angle), the reflected light becomes light having the s-polarized component of 100%.

When detecting light including biological information by the detection apparatus 3, as illustrated in FIG. 5, a part of light emitted from the light emitting unit 12 becomes a first stray light component L1 that is reflected at the first surface 51 of the cover 50 toward the light receiving unit 11, and additionally, another part becomes a second stray light component L2 that is reflected at a living body surface 70 (front surface of the measuring site M contacting the cover 50) toward the light receiving unit 11. Then, the remaining light becomes a biological passage light component L3 that is propagated in the living body (in the measuring site M) and then travels toward the light receiving unit 11. For example, the first surface 51 and the living body surface 70 correspond to the boundary surface B of FIG. 6.

When the first stray light component L1, the second stray light component L2, and the biological passage light component L3 pass through the polarizer 60, the s-polarized component is absorbed, and the p-polarized component is incident on the light receiving unit 11. Since the first stray light component L1 and the second stray light component L2 are the reflected light, ratios of the s-polarized components are large as compared to the biological passage light component L3, and ratios of the p-polarized components are small. As a result, ratios of the first stray light component L1 and the second stray light component L2 that are absorbed when pass through the polarizer 60 are large, and amounts of light incident on the light receiving unit 11 are small. In contrast, a ratio of the s-polarized component of the biological passage light component L3 is small as compared to the first stray light component L1 and the second stray light component L2, and thus, a ratio of the biological passage light component L3 that is absorbed when passing through the polarizer 60 is small. Thus, the light incident on the light receiving unit 11 becomes light in which a ratio of the biological passage light component L3 is higher than before passing through the polarizer 60, and that has less noise (the stray light component).

### Main Effects of First Exemplary Embodiment

As described above, the detection apparatus 3 of the first exemplary embodiment includes the light emitting unit 12 and the light receiving unit 11 aligned in the X direction (first direction), and the polarizer 60 that covers the +Z direction, which is the incident side of light in the light receiving unit 11. The polarizer 60 absorbs the s-polarized component. Furthermore, the polarizer 60 is the light absorbing polarizer having the absorption axis with the axial direction matching the Y direction (second direction), and covers the incident side of light (+Z direction) in the light receiving unit 11.

In addition, the detection apparatus 3 includes the optically transparent cover 50 covering the +Z direction of the light emitting unit 12 and the light receiving unit 11 (that is, the emission side of light in the light emitting unit 12, and the incident side of light in the light receiving unit 11). The polarizer 60 is disposed between the cover 50 and the light receiving unit 11.

The measuring apparatus 100 of the first exemplary embodiment includes the detection apparatus 3, and the control device 5 as the information analysis unit that identifies biological information from detection signals indicating detection results of the detection apparatus 3.

The detection apparatus 3 of the first exemplary embodiment includes the light absorbing polarizer 60 disposed on the incident side of the light receiving unit 11, and the polarizer 60 is configured such that a direction of the absorption axis is a direction in which the s-polarized light is absorbed. As a result, by utilizing the fact that the stray light component reflected by the first surface 51 of the cover 50 or the living body surface 70 becomes light having a component ratio of the s-polarized light higher than that of light passing through the living body, the large stray light component can be removed. Thus, the ratio of the stray light component (noise) in the light incident on the light receiving unit 11 can be reduced, and the ratio of the biological passage light component can be increased. Thus, detection accuracy of biological information can be enhanced.

Note that, the detection apparatus 3 of the first exemplary embodiment has the configuration including the cover 50 disposed on the incident side (+Z direction) of the polarizer 60, but may have a configuration in which the cover 50 is omitted. For example, a configuration may be adopted in which the polarizer 60 also serves as a cover. Alternatively, a configuration may be adopted in which, the polarizer 60 is disposed at a front surface of the light receiving unit 11, and the light receiving unit 11 and the polarizer 60 are sealed with the first sealing material 41. That is, it is sufficient to adopt a configuration in which the polarizer 60 absorbs a polarized component in the Y direction (second direction) in a surface covering the incident side of light (+Z direction) in the light receiving unit 11. When a wire grid polarizer is used as the polarizer 60, the wire grid polarizer can be formed by integral film formation at the front surface of the light receiving unit 11.

### 2. Second Exemplary Embodiment

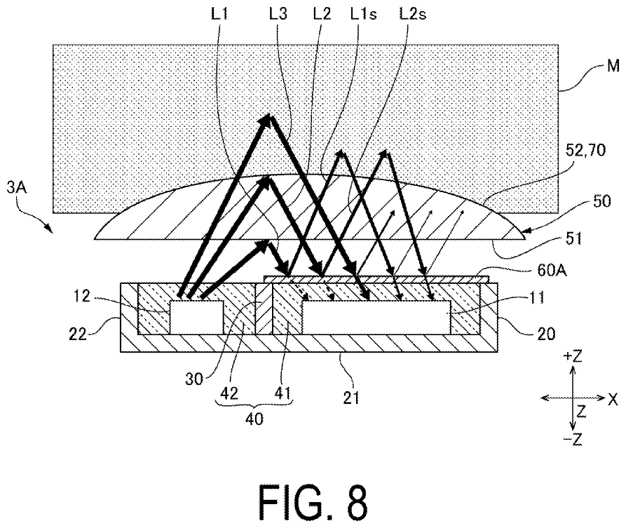

FIG. 8 is a diagram schematically illustrating a cross-sectional configuration of a detection apparatus 3A of a second exemplary embodiment, and a situation when detecting light including biological information. The detection apparatus 3A of the second exemplary embodiment differs from the first exemplary embodiment in that the polarizer 60 is changed to a polarizer 60A, and other configurations are identical to those of the first exemplary embodiment.

The polarizer 60A is disposed between the cover 50 and the light receiving unit 11. The polarizer 60A is a light reflective polarizer having a reflective axis with an axial direction matching the Y direction (second direction), and covers an incident side of light (+Z direction) in the light receiving unit 11. Thus, the polarizer 60A reflects an s-polarized component of light incident from the cover 50 side, and transmits a p-polarized component. The polarizer 60A is disposed at the front surface of the first sealing material 41, and covers the entire front surface of the first sealing material 41. Note that, as in the first exemplary embodiment, a configuration may be adopted in which, the polarizer 60A is disposed at the front surface of the light receiving unit 11, and the light receiving unit 11 and the polarizer 60A are sealed with the first sealing material 41.

As illustrated in FIG. 8, when detecting light including biological information by the detection apparatus 3A of the second exemplary embodiment, the s-polarized components of the first stray light component L1 reflected at the first surface 51 of the cover 50, and the second stray light component L2 reflected at the living body surface 70, are reflected by the polarizer 60A. Here, FIG. 8 illustrates a situation in which an s-polarized component L1s of the first stray light component L1, and an s-polarized component L2s of the second stray light component L2 are reflected by the polarizer 60A, and then parts thereof are incident on the living body, become light passing through the living body, and reach the polarizer 60A again. A part of the s-polarized component L2s becomes a p-polarized component including biological information while passing through the living body, passes through the polarizer 60A, and is incident on the light receiving unit 11. The polarizer 60A is, for example, a wire grid polarizer in which wire-shaped metal layers projecting in the Z direction are arrayed in parallel at a predetermined pitch at an optically transparent substrate or an optically transparent film. Note that, it is sufficient that the polarizer 60A is configured to reflect the s-polarized component.

As describe above, the detection apparatus 3A of the second exemplary embodiment includes the light reflective polarizer 60A disposed on the incident side of the light receiving unit 11, and the polarizer 60A is configured such that a direction of the reflective axis is a direction in which the s-polarized light is reflected. As a result, by utilizing the fact that the stray light component reflected by the first surface 51 of the cover 50 or the living body surface 70 becomes light having a component ratio of the s-polarized light higher than that of light passing through the living body, the large stray light component can be removed. In addition, the polarizer 60A does not absorb but reflects the s-polarized component, the reflected s-polarized component can be caused to enter the living body, and reused, and thus a biological passage light component incident on the light receiving unit 11 can be increased. Thus, a ratio of the biological passage light component incident on the light receiving unit 11 can be further increased as compared to the first exemplary embodiment. Thus, it is possible to further enhance the detection accuracy of the biological information as compared to the first exemplary embodiment.

### 3. Third Exemplary Embodiment

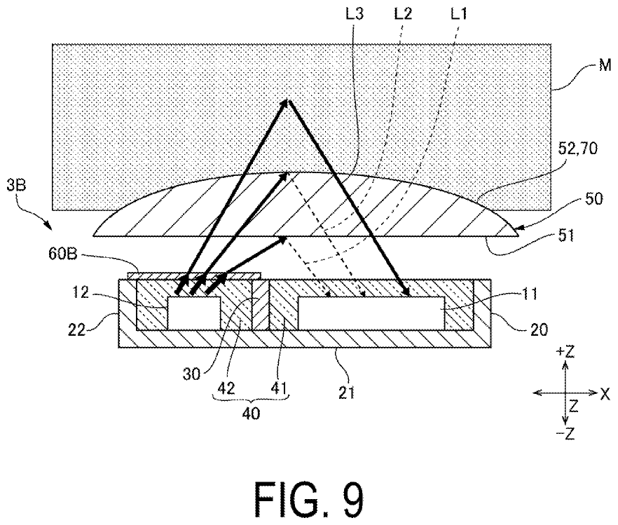

FIG. 9 is a diagram schematically illustrating a cross-sectional configuration of a detection apparatus 3B of a third exemplary embodiment, and a situation when detecting light including biological information. The detection apparatus 3B of the third exemplary embodiment differs from the first exemplary embodiment in that the polarizer 60 is changed to a polarizer 60B, and other configurations are identical to those of the first exemplary embodiment.

The polarizer 60B is disposed between the cover 50 and the light emitting unit 12. The polarizer 60B is a light absorbing polarizer having an absorption axis with an axial direction matching the Y direction, and covers an emission side of light (+Z direction) in the light emitting unit 12. Thus, the polarizer 60B absorbs an s-polarized component of light emitted from the light emitting unit 12 and transmits a p-polarized component. The polarizer 60B is disposed at the front surface of the second sealing material 42, and covers the entire front surface of the second sealing material 42. Note that, as in the first exemplary embodiment, a configuration may be adopted in which, the polarizer 60B is disposed at a front surface of the light emitting unit 12, and the light emitting unit 12 and the polarizer 60B are sealed with the second sealing material 42.

As illustrated in FIG. 9, when detecting light including biological information by the detection apparatus 3B of the third exemplary embodiment, the s-polarized component is removed from the light emitted from the light emitting unit 12. Therefore, since the p-polarized component with small reflectance reaches the first surface 51 of the cover 50 and the living body surface 70, amounts of light of the first stray light component L1, which is reflected by the first surface 51, and the second stray light component L2, which is reflected by the living body surface 70 are small.

As described above, the detection apparatus 3B of the third exemplary embodiment includes the light absorbing polarizer 60B disposed on the emission side of the light emitting unit 12, and the polarizer 60B is configured such that a direction of the absorption axis is a direction in which the s-polarized light is absorbed. As a result, the s-polarized component can be removed from the light emitted from the light emitting unit 12, and thus amounts of light of the first stray light component L1 and the second stray light component L2 that do not include biological information can be reduced by using the small reflectance of the p-polarized component. Therefore, it is possible to increase a ratio of a biological passage light component in light incident on the light receiving unit 11, and thus detection accuracy of biological information can be enhanced.

### 4. Fourth Exemplary Embodiment

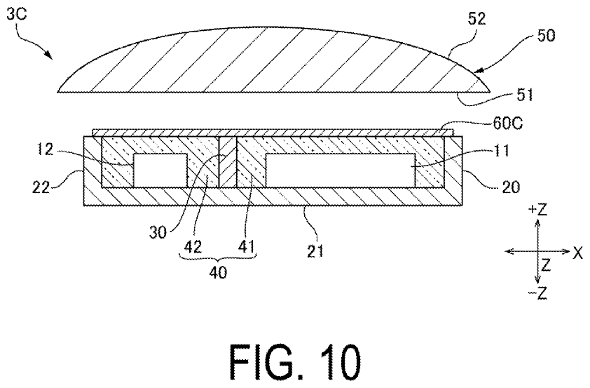

FIG. 10 is a diagram schematically illustrating a cross-sectional configuration of a detection apparatus 3C of a fourth exemplary embodiment. The detection apparatus 3C of the fourth exemplary embodiment differs from the first exemplary embodiment in that the polarizer 60 is changed to a polarizer 60C, and other configurations are identical to those of the first exemplary embodiment. The polarizer 60C is obtained by integrating the polarizer 60 of the first exemplary embodiment and the polarizer 60B of the third exemplary embodiment.

The polarizer 60B is disposed between the cover 50, and the light emitting unit 12 and the light receiving unit 11. The polarizer 60B covers both the light emitting unit 12 and the light receiving unit 11 from the +Z direction (the emission side of light in the light emitting unit 12, and the incident side of light in the light receiving unit 11). The polarizer 60C is disposed at front surfaces of the first sealing material 41 and the second sealing material 42, and covers the entire front surfaces of the first sealing material 41 and the second sealing material 42. Note that, a configuration may be adopted in which, the polarizer 60C is divided into two parts, and one of the divided polarizers 60 is disposed at the front surface of the light receiving unit 11 and sealed with the first sealing material 41, and another of the divided polarizers 60 is disposed at the front surface of the light emitting unit 12 and sealed with the second sealing material 42.

The polarizer 60C is a light absorbing polarizer having an absorption axis with an axial direction matching the Y direction. Thus, the polarizer 60C absorbs an s-polarized component of light emitted from the light emitting unit 12 and transmits a p-polarized component. Also, the polarizer 60C absorbs an s-polarized component of light incident on the light receiving unit 11 and transmits a p-polarized component.

When detecting light including biological information by the detection apparatus 3C of the fourth exemplary embodiment, in the same manner as in the third exemplary embodiment, the s-polarized component is removed from the light emitted from the light emitting unit 12, and the p-polarized component with small reflectance reaches the first surface 51 of the cover 50 and the living body surface 70. As a result, amounts of light of the first stray light component L1 and the second stray light component L2 that do not include biological information can be reduced by using the small reflectance of the p-polarized component. Furthermore, the first stray light component L1, the second stray light component L2, and the biological passage light component L3 pass through the polarizer 60 before reaching the light receiving unit 11 in the same manner as in the first exemplary embodiment. At this time, since the s-polarized component is absorbed, the s-polarized component generated by reflection can be removed from the first stray light component L1 and the second stray light component L2. Thus, amounts of light of the first stray light component L1 and the second stray light component L2 incident on the light receiving unit 11 can be further reduced. Therefore, it is possible to increase a ratio of a biological passage light component in light incident on the light receiving unit 11, and thus detection accuracy of biological information can be enhanced.

### 5. Fifth Exemplary Embodiment

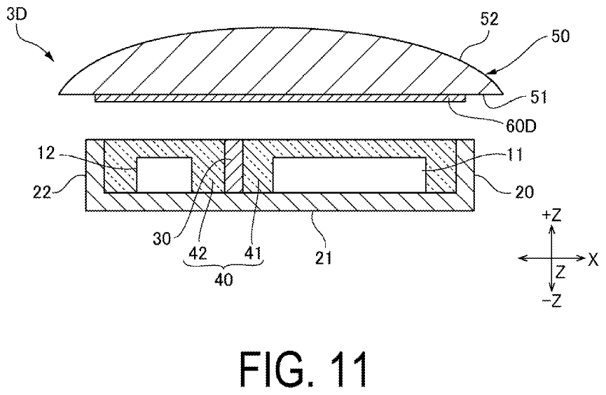

FIG. 11 is a diagram schematically illustrating a cross-sectional configuration of a detection apparatus 3D of a fifth exemplary embodiment. The detection apparatus 3D of the fifth exemplary embodiment differs from the fourth exemplary embodiment in that a polarizer 60D is fixed to the cover 50 instead of a front surface of the sealing material 40, and other configurations are the same as those of the fourth exemplary embodiment. The polarizer 60D is disposed at the first surface 51 of the cover 50. The first surface 51 is a front surface facing the light receiving unit 11 and the light emitting unit 12. For example, the polarizer 60D is fixed to the first surface 51 by bonding. The polarizer 60D is a light absorbing polarizer having an absorption axis with an axial direction matching the Y direction.

With the detection apparatus 3D of the fifth exemplary embodiment, the same action effect as the fourth exemplary embodiment can be obtained. That is, amounts of light of the first stray light component L1 and the second stray light component L2 that do not include biological information can be reduced by using small reflectance of a p-polarized component. Furthermore, an s-polarized component caused by reflection can be removed from the first stray light component L1 and the second stray light component L2. Therefore, it is possible to increase a ratio of a biological passage light component in light incident on the light receiving unit 11, and thus detection accuracy of biological information can be enhanced.

Note that, even in the first exemplary embodiment, the second exemplary embodiment, and the third exemplary embodiment, a configuration can be adopted in which the polarizer is disposed at the first surface 51 of the cover 50.

### 6. Modified Examples

(1) In each of the above-described exemplary embodiments, the second surface 52 of the cover 50 is the protruding curved surface, but the second surface 52 may be a flat surface perpendicular to the Z direction.
(2) In the first exemplary embodiment to the fourth exemplary embodiment, an air layer is provided between the first surface 51 of the cover 50 and the sealing material 40, but the sealing material 40 may be filled to eliminate the air layer. Similarly, in the fifth exemplary embodiment, an air layer is provided between the polarizer 60D fixed to the first surface 51 and the sealing material 40, but the sealing material 40 may be filled to eliminate the air layer.

What is claimed is:

1. A detection apparatus, comprising:
a light emitting unit;
a light receiving unit aligned with the light emitting unit in a first direction; and
a polarizer configured to cover at least one of an emission side of light in the light emitting unit, and an incident side of light in the light receiving unit, wherein
the polarizer absorbs or reflects an s-polarized component.

2. The detection apparatus according to claim 1, comprising:
an optical transparent cover configured to cover an emission side of light in the light emitting unit, and an incident side of light in the light receiving unit, wherein
the polarizer is disposed at least one of between the cover and the light emitting unit, and between the cover and the light receiving unit.

3. The detection apparatus according to claim 2, wherein
the polarizer is disposed at a front surface of the cover.

4. The detection apparatus according to claim 1, wherein
the polarizer is disposed at at least one of a front surface of the light emitting unit, and a front surface of the light receiving unit.

5. The detection apparatus according to claim 1, wherein
the polarizer absorbs or reflects a polarized component in a second direction intersecting the first direction, on a surface covering at least one of an emission side of light in the light emitting unit, and an incident side of light in the light receiving unit.

6. The detection apparatus according to claim 5, wherein
the polarizer is a light reflective polarizer having a reflective axis with an axial direction matching the second direction, and covers an incident side of light in the light receiving unit.

7. The detection apparatus according to claim 5, wherein
the polarizer is a light absorbing polarizer having an absorption axis with an axial direction matching the second direction, and covers an incident side of light in the light receiving unit.

8. The detection apparatus according to claim 5, wherein
the polarizer is a light absorbing polarizer having an absorption axis with an axial direction matching the second direction, and covers an emission side of light in the light emitting unit.

9. The detection apparatus according to claim 5, wherein
the polarizer is a light absorbing polarizer having an absorption axis with an axial direction matching the second direction, and covers an emission side of light in the light emitting unit, and an incident side of light in the light receiving unit.

10. A measuring apparatus comprising:
the detection apparatus according to claim 1; and
an information analysis unit configured to identify biological information from a detection signal indicating a detection result of the detection apparatus.
